(19) United States
(12) Patent Application Publication
(10) Pub. No.: US 2023/0015643 A1
(43) Pub. Date: Jan. 19, 2023

(54) SYSTEMS AND METHODS FOR FACILITATING REHABILITATIVE EXERCISE COMPLIANCE

(71) Applicant: Vive Health LLC, Naples, FL (US)

(72) Inventors: Anthony Henry Barbier, Bonita Springs, FL (US); Aaron B. Gaunt, Estero, FL (US)

(21) Appl. No.: 17/374,935

(22) Filed: Jul. 13, 2021

(51) Int. Cl. G16H 20/30; A63B 24/00; G16H 40/67; G16H 15/00

(52) U.S. Cl. CPC G16H 20/30; A63B 24/0062; G16H 40/67; G16H 15/00; A63B 2024/0068; A63B 2220/17

(57) ABSTRACT

A system for facilitating compliance with a rehabilitative exercise regimen is configurable to access a rehabilitative exercise regimen comprising a plurality of rehabilitative exercises. The plurality of rehabilitative exercises includes at least a first rehabilitative exercise and a second rehabilitative exercise. The system is further configurable to obtain exercise analysis output. The exercise analysis output is based on sensor data capturing a user performance of a user performing the first rehabilitative exercise. The system is further configurable to, in response to determining that the exercise analysis output indicates satisfactory completion of the first rehabilitative exercise by the user, configure an exercise interface to permit the user to proceed to the second rehabilitative exercise.

### BACKGROUND

Individuals engage in rehabilitative exercises for a variety of purposes. For example, disorders, injury, and/or bodily trauma can cause individuals to experience reduced bodily capabilities, such as reduced range of motion, reduced strength, etcetera. To restore muscle, range of motion, and/or other capabilities, such individuals may undergo one or more rehabilitative exercises.

In some instances, a medical practitioner prescribes a rehabilitative exercise regimen to a patient to assist the patient in pursuing rehabilitation objectives. A rehabilitative exercise regimen can include a series of ordered exercises that build on one another to increase the physical capabilities of the individual. For example, an initial exercise may be tailored to assist an individual in re-establishing an initial range of motion, and a subsequent exercise may be tailored to further assist the individual in expanding the initial range of motion to an increased range of motion.

Individuals who are prescribed rehabilitative exercise regimens often perform at least some of the exercises without in-person supervision by a medical practitioner. Although this lack of supervision provides individuals with greater autonomy, such a lack of supervision allows many individuals to fail to appropriately comply with a prescribed rehabilitative exercise regimen. For example, individuals may fail to perform exercises correctly, such as by failing to perform an adequate number of repetitions, failing to hold positions for an adequate amount of time, etcetera. As another example, individuals may fail to perform exercises according to a prescribed sequence, ordering, and/or timing. For instance, an individual may proceed to a subsequent exercise of an exercise regimen without first adequately establish their bodily capabilities by correctly performing a preceding exercise of the exercise regimen. Prematurely performing more advanced exercises in this manner can lead to injury and/or degraded patient outcomes.

For at least the foregoing reasons, there is an ongoing need and desire for improved systems and methods for facilitating rehabilitative exercise compliance.

The subject matter claimed herein is not limited to embodiments that solve any disadvantages or that operate only in environments such as those described above. Rather, this background is only provided to illustrate one exemplary technology area where some embodiments described herein may be practiced.

### BRIEF SUMMARY

Implementations of the present disclosure extend at least to systems and methods for facilitating rehabilitative exercise compliance.

Embodiments include systems, methods, and hardware storage devices that are associated with the performance of various acts. The acts include accessing a rehabilitative exercise regimen comprising a plurality of rehabilitative exercises. The plurality of rehabilitative exercises includes at least a first rehabilitative exercise and a second rehabilitative exercise. The acts further include obtaining exercise analysis output. The exercise analysis output is based on sensor data capturing a user performance of a user performing the first rehabilitative exercise. The acts further include, in response to determining that the exercise analysis output indicates satisfactory completion of the first rehabilitative exercise by the user, configuring an exercise interface to permit the user to proceed to the second rehabilitative exercise.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

Additional features and advantages will be set forth in the description which follows, and in part will be obvious from the description, or may be learned by the practice of the teachings herein. Features and advantages of the invention may be realized and obtained by means of the instruments and combinations particularly pointed out in the appended claims. Features of the present invention will become more fully apparent from the following description and appended claims or may be learned by the practice of the invention as set forth hereinafter.

### BRIEF DESCRIPTION OF THE DRAWINGS

In order to describe the manner in which the above-recited and other advantages and features can be obtained, a more particular description of the subject matter briefly described above will be rendered by reference to specific embodiments which are illustrated in the appended drawings. Understanding that these drawings depict only typical embodiments and are not therefore to be considered to be limiting in scope, embodiments will be described and explained with additional specificity and detail through the use of the accompanying drawings.

FIG. 1 illustrates example components of a system for facilitating rehabilitative exercise compliance;

FIG. 2 illustrates an example user interface displaying a representation of a rehabilitative exercise regimen;

FIG. 3A illustrates a conceptual representation of a system responding to a user performing a rehabilitative exercise in a manner that differs from an expected performance of the rehabilitative exercise;

FIG. 3B illustrates a conceptual representation of a system responding to a user performing a rehabilitative exercise in a manner that conforms to an expected performance of the rehabilitative exercise;

FIG. 4 illustrates an example user interface displaying a representation of a rehabilitative exercise regimen with an additional rehabilitative exercise enabled;

FIG. 5 illustrates a conceptual representation of a system responding to a user performing the additional rehabilitative exercise in a manner that differs from an expected performance of the additional rehabilitative exercise;

FIG. 6 illustrates an example user interface displaying a representation of a rehabilitative exercise regimen with another rehabilitative exercise enabled; and

FIG. 7 illustrates an example flow diagram depicting acts associated with facilitating rehabilitative exercise compliance.

### DETAILED DESCRIPTION

Implementations of the present disclosure extend at least to systems and methods for facilitating rehabilitative exercise compliance.

Embodiments include systems, methods, and hardware storage devices that are associated with the performance of various acts. The acts include accessing a rehabilitative exercise regimen comprising a plurality of rehabilitative exercises. The plurality of rehabilitative exercises includes at least a first rehabilitative exercise and a second rehabilitative exercise. The acts further include obtaining exercise analysis output. The exercise analysis output is based on sensor data capturing a user performance of a user performing the first rehabilitative exercise. The acts further include, in response to determining that the exercise analysis output indicates satisfactory completion of the first rehabilitative exercise by the user, configuring an exercise interface to permit the user to proceed to the second rehabilitative exercise.

Those skilled in the art will recognize, in view of the present disclosure, that at least some of the disclosed embodiments may address various challenges associated with facilitating rehabilitative exercise compliance. For example, by ensuring proper compliance with a rehabilitative exercise before permitting users to proceed to a subsequent rehabilitative exercise (e.g., a more advanced rehabilitative exercise that builds on a preceding rehabilitative exercise) users may be prevented from prematurely attempting advanced exercises that are beyond their physical capabilities. In this way, users undergoing a rehabilitative exercise regimen may avoid injury or slowed/ineffective rehabilitation that may result from incorrect performance of rehabilitative exercises and/or incorrect progression through a series of rehabilitative exercises. According to the present disclosure, such benefits may be advantageously realized without in-person or direct supervision of a medical practitioner.

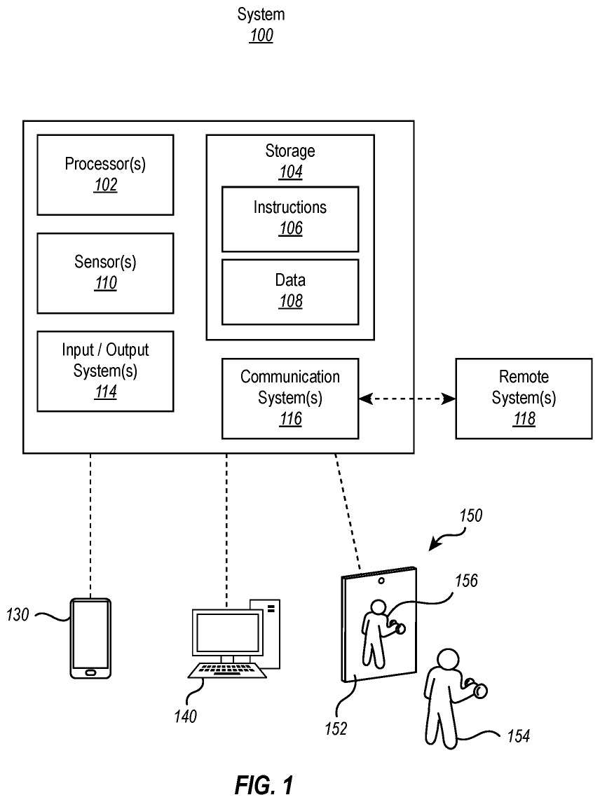

Having described some of the various high-level features and benefits of the disclosed embodiments, attention will now be directed to FIGS. 1 through 7. These Figures provide various supporting illustrations related to the disclosed embodiments.

FIG. 1 illustrates various example components of a system 100 for facilitating rehabilitative exercise compliance, in accordance with the present disclosure. For example, FIG. 1 illustrates that a system 100 may include processor(s) 102, storage 104, sensor(s) 110, input/output system(s) 114 (I/O system(s) 114), and communication system(s) 116. Although FIG. 1 illustrates a system 100 as including particular components, one will appreciate, in view of the present disclosure, that a system 100 may comprise any number of additional or alternative components.

The processor(s) 102 may comprise one or more sets of electronic circuitry that include any number of logic units, registers, and/or control units to facilitate the execution of computer-readable instructions (e.g., instructions that form a computer program). Such computer-readable instructions may be stored within storage 104. The storage 104 may comprise physical system memory and may be volatile, non-volatile, or some combination thereof. Furthermore, storage 104 may comprise local storage, remote storage (e.g., accessible via communication system(s) 116 or otherwise), or some combination thereof. Additional details related to processors (e.g., processor(s) 102) and computer storage media (e.g., storage 104) will be provided hereinafter.

As will be described in more detail, the processor(s) 102 may be configured to execute instructions 106 stored within storage 104 to perform certain actions associated with facilitating rehabilitative exercise compliance. The actions may rely at least in part on data 108 stored on storage 104 in a volatile or non-volatile manner.

In some instances, the actions may rely at least in part on communication system(s) 116 for receiving data and/or instructions from remote system(s) 118, which may include, for example, separate systems or computing devices, storage nodes, sensors, and/or others. The communications system(s) 118 may comprise any combination of software or hardware components that are operable to facilitate communication between on-system components/devices and/or with off-system components/devices. For example, the communications system(s) 118 may comprise ports, buses, or other physical connection apparatuses for communicating with other devices/components. Additionally, or alternatively, the communications system(s) 118 may comprise systems/components operable to communicate wirelessly with external systems and/or devices through any suitable communication channel(s), such as, by way of non-limiting example, Bluetooth, ultra-wideband, Wi-Fi, WLAN, infrared communication, and/or others.

FIG. 1 illustrates that a system 100 may comprise or be in communication with sensor(s) 110. Sensor(s) 110 may comprise any device for capturing or measuring data representative of perceivable phenomenon. By way of non-limiting example, the sensor(s) 110 may comprise one or more image sensors, optical scanners, microphones, thermometers, barometers, magnetometers, accelerometers, gyroscopes, and/or others. In some implementations, the sensor(s) 110 include systems/devices capable of sensing data representative of physical actions performed by a user. For example, the sensor(s) 110 may include one or more depth sensors or other capture devices for detecting bodily position and/or motion as a user performs a rehabilitative exercise. Additionally, or alternatively, the sensor(s) 110 may include one or more heartrate monitors, oximeters, electromyographs, and/or other devices for measuring data associated with bodily activity/function.

Furthermore, FIG. 1 illustrates that a system 100 may comprise or be in communication with I/O system(s) 114. I/O system(s) 114 may include any type of input or output device such as, by way of non-limiting example, a display, a touch screen, a mouse, a keyboard, a controller, a speaker, a light source, a vibration motor, and/or others, without limitation. In some instances, I/O system(s) 114 may at least partially rely on the sensor(s) 110 (e.g., via a microphone or motion sensor system to detect user input).

FIG. 1 conceptually represents that the components of the system 100 may comprise or utilize various types of devices, such as mobile electronic device 130 (e.g., a smartphone), personal computing device 140, and/or other devices more specifically tailored to assisting users in the performance of exercises, such as exercise system 150. An exercise system 150 can include an exercise interface 152 (e.g., a display) for presenting exercise activities and/or information for viewing by a user 154. For example, an exercise interface 152 of an exercise system 150 may display a representation of a virtual instructor 156 performing a particular type of exercise, and the user 154 may mimic, emulate, and/or follow directions of the virtual instructor 156 to attempt the particular type of exercise. Although the present description focuses, in at least some respects, on utilizing an exercise system 150 to implement techniques of the present disclosure, additional or alternative types of systems may be used.

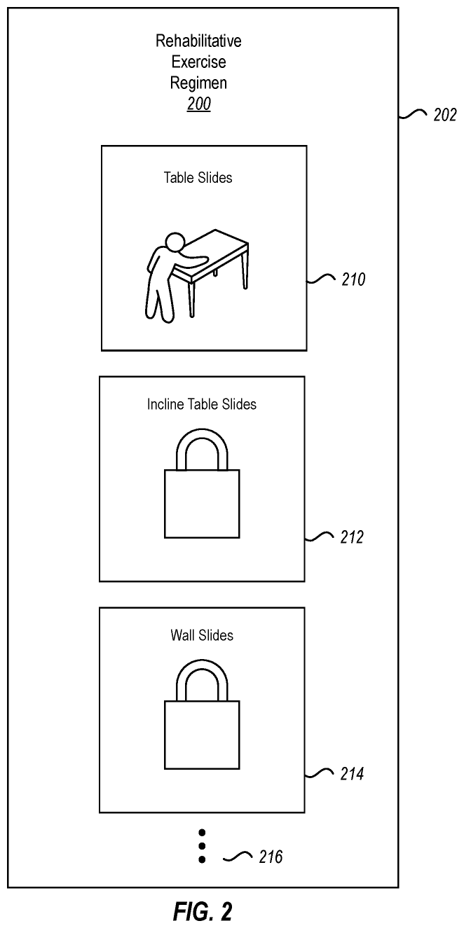

FIG. 2 illustrates an example user interface 202 (e.g., of a system 100) displaying a representation of a rehabilitative exercise regimen 200. Aspects of the user interface 202 as illustrated in FIG. 2 may be displayed, for example, on an exercise interface of an exercise system or other device for viewing and/or interaction by a user.

The rehabilitative exercise regimen 200 illustrated in FIG. 2 includes various individual rehabilitative exercises represented by tiles or objects within the user interface 202. In the example shown in FIG. 2, the rehabilitative exercise regimen 200 includes a first rehabilitative exercise 210, a second rehabilitative exercise 212, and a third rehabilitative exercise 214. The first rehabilitative exercise 210 corresponds to table slides, the second rehabilitative exercise 212 corresponds to incline table slides, and the third rehabilitative exercise 214 corresponds to wall slides. FIG. 2 also provides an ellipsis 216 indicating that the rehabilitative exercises 210, 212, 214 of the rehabilitative exercise regimen 200 of FIG. 2 are provided as an example only and are not limiting of the present disclosure. A rehabilitative exercise regimen 200 may include any number of additional or alternative rehabilitative exercises, in accordance with the present disclosure (indicated in FIG. 2 by the ellipsis 216).

As used herein, a “rehabilitative exercise” refers to any type of exercise designed to restore or improve an individual's bodily capabilities, such as range of motion, strength, stability, motor control, etc. Such rehabilitative exercises may be appropriate, for example, when a user experiences a bodily disorder, injury, and/or bodily trauma that reduces their bodily capabilities from an initial or expected level to a reduced or degraded level. For instance, the first rehabilitative exercise 210 of FIG. 2, table slides, may involve an individual standing next to a tabletop or counter with their arm resting on the tabletop or counter. The individual may relax their shoulder and neck muscles and slowly lean forwards and slightly outward from the individual's sagittal plane (e.g., at a 45-degree angle) while sliding the individual's hand on the tabletop or counter. When a mild stretch is felt, the individual may pause and hold the position for five to ten seconds. Table slides may be beneficially performed by individuals who experiences a rotator cuff injury (e.g., a rotator cuff tear) and/or rotator cuff treatment (e.g., arthroscopic surgery to repair torn tendons) and may assist in rebuilding range of motion and/or strength of rotator cuff muscles.

As used herein, a “rehabilitative exercise regimen” includes a plurality of rehabilitative exercises that build on one another to facilitate restoration of bodily capabilities. For instance, rehabilitative exercises of a rehabilitative exercise regimen can be associated with respective rehabilitative progression levels, and different rehabilitative exercises can be associated with higher or lower rehabilitative progression levels relative to one another. For instance, the first rehabilitative exercise 210 of FIG. 2, table slides, requires low activation of rotator cuff muscles and can therefore often be performed immediately after an initial immobilization period following a rotator cuff surgery (e.g., after 4 to 6 weeks). The first rehabilitative exercise 210 of the rehabilitative exercise regimen 200 may thus be associated with an initial, base, or first rehabilitative progression level. As used herein, a “rehabilitative progression level” for a particular rehabilitative exercise refers to a level of bodily capability (e.g., for a particular body part, such as a rotator cuff) that should exist prior to the performance of the particular rehabilitative exercise (e.g., in order to prevent injury, facilitate ideal rehabilitation, attain expected bodily progression from performance of the particular rehabilitative exercise, etc.).

In contrast, the second rehabilitative exercise 212 of FIG. 2, incline table slides, lead to greater activation of rotator cuff muscles than ordinary table slides (i.e., the first rehabilitative exercise 210 of FIG. 2) and are typically prescribed for performance after an individual has sufficiently built underlying strength in the rotator cuff muscles by performing the first rehabilitative exercise 210 and/or other rehabilitative exercises associated with base or initial rehabilitative progression levels. The second rehabilitative exercise 212 of the rehabilitative exercise regimen 200 may thus be associated with an intermediate or second rehabilitative progression level (greater than the initial or first rehabilitative progression level associated with the first rehabilitative exercise 210).

The second rehabilitative exercise 212, incline table slides, may involve an individual standing with their hand resting on an inclined surface (e.g., inclined by about 45 degrees). The individual relaxes their shoulder and neck muscles while sliding their hand up the incline. At the top of the incline, the individual holds the position for two to three seconds.

Similarly, the third rehabilitative exercise 214 of FIG. 2, wall slides, leads to still greater activation of rotator cuff muscles than incline table slides (i.e., the second rehabilitative exercise 212 of FIG. 2) and are typically prescribed for performance after an individual has sufficiently built underlying strength in the rotator cuff muscles by performing the first rehabilitative exercise 210 and, subsequently, the second rehabilitative exercise 212. In this way, the third rehabilitative exercise 214 of the rehabilitative exercise regimen 200 may be associated with a greater or third rehabilitative progression level (greater than the intermediate or second rehabilitative progression level associated with the second rehabilitative exercise 212).

The third rehabilitative exercise 214, wall slides, may involve an individual transitioning toward raising their arm above their head (e.g., following a rotator cuff injury or medical operation. For instance, the individual may begin by standing in front of a wall with their shoulder and neck muscles relaxed. The individual may then slide their hand up the wall and hold the position with their hand extended up the wall for two or three seconds. The individual may then lower their arm in a slow, controlled manner by sliding their hand back down the wall. Initially, the individual may use their uninvolved hand to assist in the performance of wall slides.

In view of the foregoing example, a rehabilitative exercise regimen 200 may include multiple rehabilitative exercises (e.g., 210, 212, 214) that build on one another and are specifically tailored to the restoration of bodily capabilities for one or more particular body parts (e.g., the rotator cuff, according to the foregoing example). For instance, a first rehabilitative exercise of a rehabilitative exercise regimen 200 may be associated with a first rehabilitative progression level that is lower than a second rehabilitative progression level associated with a second rehabilitative exercise of the rehabilitative exercise regimen. Stated differently, performance of a second rehabilitative exercise may rely on completed performance of the first rehabilitative exercise to sufficiently build bodily capabilities that should exist prior to performance of the second rehabilitative exercise (e.g., to render performance of the second rehabilitative exercise beneficial for patient rehabilitation). In some instances, premature performance of the second rehabilitative exercise (e.g., prior to sufficiently building bodily capabilities via performance of the first rehabilitative exercise) can lead to injury and/or degraded patient outcomes.

In this way, a rehabilitative exercise regimen differs from a set of conventional strength training or cardiovascular training exercises that are not dependent on and do not build on one another, with regard to bodily rehabilitation. A rehabilitative exercise regimen, according to the present disclosure, may omit ordinary strength training and/or cardiovascular training exercises that do not specifically build on one another to facilitate bodily rehabilitation (e.g., where the performance of a first exercise is tailored to increase bodily capabilities, such as range of motion, muscular strength or control, etc., to a recommended level that should exist prior to the performance of a second exercise to render performance of the second exercise optimally beneficial or safe).

Although the foregoing example focuses, in at least some respects, on single rehabilitative exercises that are associated with different rehabilitative progression levels, one will appreciate, in view of the present disclosure, that a rehabilitative exercise regimen can include separate sets of rehabilitative exercises, where one set is associated with a higher rehabilitative progression level than a second set.

FIG. 2 illustrates the second rehabilitative exercise 212 and the third rehabilitative exercise 214 of the rehabilitative exercise regimen 200 in a locked state, indicated by the representation of the locks presented on the user interface 202 in association with the second and third rehabilitative exercises 212, 214. In some instances, the second and third rehabilitative exercises 212, 214 are presented in a locked state before bodily capabilities (e.g., rotator cuff strength and/or range of motion) of a user have been sufficiently established by performance of the first rehabilitative exercise 210 to render performance of the second and/or third rehabilitative exercises 212, 214 safe and/or beneficial. As will be described in more detail hereinbelow, a system (e.g., system 100) may facilitate compliance with the example rehabilitative exercise regimen 200 by preventing access to the second and third rehabilitative exercises 212, 214 until satisfactory performance of the first rehabilitative exercise has been detected.

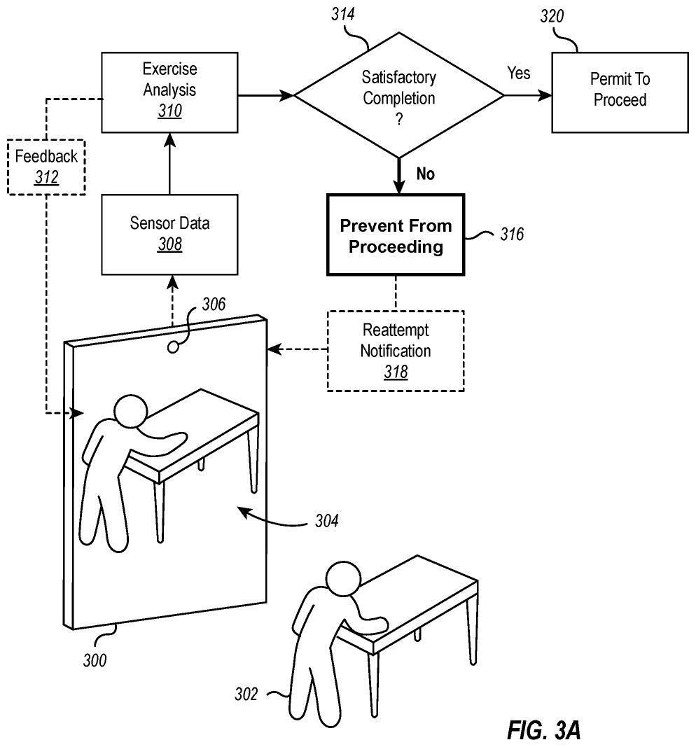

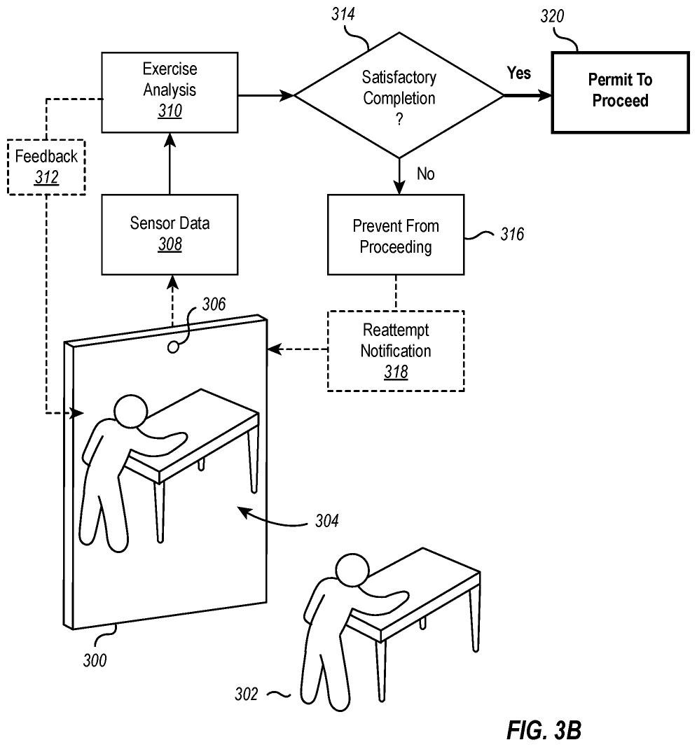

FIG. 3A illustrates a conceptual representation of a system 300 responding to a user 302 performing the first rehabilitative exercise 210 (i.e., table slides) in a manner that differs from an expected performance of the first rehabilitative exercise 210. The system 300 may correspond, in at least some respects, to the system 100 described hereinabove with reference to FIG. 1. For example, the system 300 may include an exercise interface 304 that presents exercise information to the user 302, such as representation of a rehabilitative exercise regimen 200 (e.g., from FIG. 2) or a representation of an expected performance of a particular rehabilitative exercise. FIG. 3A shows an example in which the exercise interface 304 displays a representation of an expected performance of the first rehabilitative exercise 210, table slides, showing a correct manner in which table slides should be performed to achieve ideal patient outcomes. The system 300 may also include one or more sensors 306 (e.g., optical or other computer vision systems) configured to capture sensor data 308 representative of a user attempting to perform exercise activities (e.g., performing the first rehabilitative exercise 210).

FIG. 3A furthermore illustrates that a system 300 can generate or obtain exercise analysis output 310 based on the sensor data 308 capturing the user performance of the first rehabilitative exercise 210. In some implementations, the exercise analysis output 310 includes a comparison between the user performance of the first rehabilitative exercise 210 represented by the captured sensor data 308 and an expected performance of the first rehabilitative exercise 210 (e.g., according to accepted rehabilitation standards). Such a comparison may indicate whether the user 302 performed the first rehabilitative exercise 210 in a manner that builds bodily capabilities and/or pursues rehabilitative goals.

A comparison between a user performance and an expected performance may be facilitated, in some instances, utilizing smart motion technology (SMT) and/or artificial intelligence (AI) algorithms to compare attributes from the user performance and the expected performance. Compared attributes may include, by way of non-limiting example, number of repetitions, amount of time in an isometric position, form, and/or others.

For instance, the exercise analysis output 310 may compare a detected number of repetitions from the user performance (e.g., based on the sensor data 308) to an expected number of repetitions associated with the expected performance to determine whether the user performance complies with the expected performance. For example, the sensor data 308 may indicate that the user 302 has performed two repetitions of table slides during an exercise session, whereas an expected number of table slide repetitions is three for an exercise session. In such an example, the exercise analysis output 310 may indicate the user 302 has failed to complete the first rehabilitative exercise 210 as expected.

Additionally, or alternatively, the exercise analysis output 310 may compare a detected amount of time in an isometric position from the user performance and an expected amount of time in the isometric position associated with the expected performance to determine whether the user performance complies with the expected performance. For example, the sensor data 308 may indicate that the user 302 has held a stretched position with their hand slid outward over the tabletop for a total of four seconds, whereas the expected amount of time spent in such positioning is five to ten seconds. In such an example, the exercise analysis output 310 may indicate the user 302 has failed to complete the first rehabilitative exercise 210 as expected.

In some instances, the exercise analysis output 310 may compare a detected form from the user performance and an expected form associated with the expected performance to determine whether the user performance complies with the expected performance, such as where a rehabilitative exercise includes fluid motions and/or static poses. In some implementations, computer vision techniques may be employed to detect motion paths and/or bodily poses of the user 302 while performing the first rehabilitative exercise 210, and satisfactory performance of the first rehabilitative exercise 210 may be determined based on whether threshold similarities are satisfied between the detected motion paths and/or bodily poses and the expected motion paths and/or bodily poses. For example, the sensor data 308 may indicate that the user 302 failed to adequately lean forward over the tabletop, as compared to an expected or proper performance of a table slide, or that the user 302 extended their hand forward over the table in alignment with their sagittal plane rather than slightly outward from their sagittal plane (e.g., at a 45-degree angle). In such examples, the exercise analysis output 310 may indicate the user 302 has failed to complete the first rehabilitative exercise 210 as expected.

A comparison between a user performance and an expected performance of a rehabilitative exercise may include any combination of the foregoing and/or other exercise attributes. For example, whether a user repetition of an exercise is counted may depend on the form the user exhibited during the repetition, or an amount of time a user spent in an isometric position may correspond to the amount of time the user spent in the isometric position with proper form. Furthermore, different aspects of an analyzed user performance of a rehabilitative exercise may be weighted differently for determining whether the user performance complies with an expected performance (e.g., for some exercises, proper form may be weighted more than repetitions performed or time spent holding a stretch/position).

In some implementations, upon detecting deficiencies in the user performance based on the exercise analysis output 310, the system 300 may provide feedback 312 at the exercise interface 304 detailing which aspects of the user's performance the user 302 may modify to conform to an expected performance of a rehabilitative exercise (e.g., directing the user 302 to hold a position longer, to perform additional repetitions, to modify their form in one or more particular ways, etc.).

As noted above, the exercise analysis output 310 may indicate whether a user 302 has satisfactorily completed a rehabilitative exercise (e.g., the first rehabilitative exercise 210), which is illustrated in FIG. 3A by decision block 314. As described above, the user 302 has failed to satisfactorily complete the first rehabilitative exercise 210, as further emphasized in FIG. 3A by the word “No” provided in bold format and extending from decision block 314. Block 316 of FIG. 3A (also provided in bold format) illustrates that, in response to determining that the exercise analysis output 310 indicates unsatisfactory completion of the first rehabilitative exercise 210 by the user 302, the system 300 may prevent the user 302 from proceeding to subsequent rehabilitative exercises (e.g., the second rehabilitative exercise 212). For example, the system 300 may maintain a locked state for the second rehabilitative exercise 212 (see FIG. 2) that prevents the user 302 from initiating and/or logging progress toward completion of the second rehabilitative exercise 212 on the exercise interface 304.

In some implementations, the system 300 may be configured to provide, at the exercise interface 304, a reattempt notification 318 that indicates that the user 302 has not completed the first rehabilitative exercise 210 in a satisfactory manner and needs to reattempt the first rehabilitative exercise 210 (e.g., in order to progress toward completion of the rehabilitative exercise regimen 200 and/or the other rehabilitative exercises thereof).

If, on the other hand, the system 300 determines that the user's performance of the first rehabilitative exercise 210 is satisfactory, the system 300 may permit the user 302 to proceed to subsequent rehabilitative exercises (indicated in FIGS. 3A and 3B by block 320). FIG. 3B illustrates a conceptual representation of the system 300 responding to the user 302 performing the first rehabilitative exercise 210 in a manner that conforms to the expected performance of the first rehabilitative exercise 210. For example, the exercise analysis output 310 may indicate (based on sensor data 308) that the user 302 performed an adequate number of table slide repetitions, extended their hand over the tabletop in the proper direction (e.g., outward from the sagittal plane of the user 302), and/or held appropriate positioning over the tabletop for a sufficient amount of time (e.g., five to ten seconds).

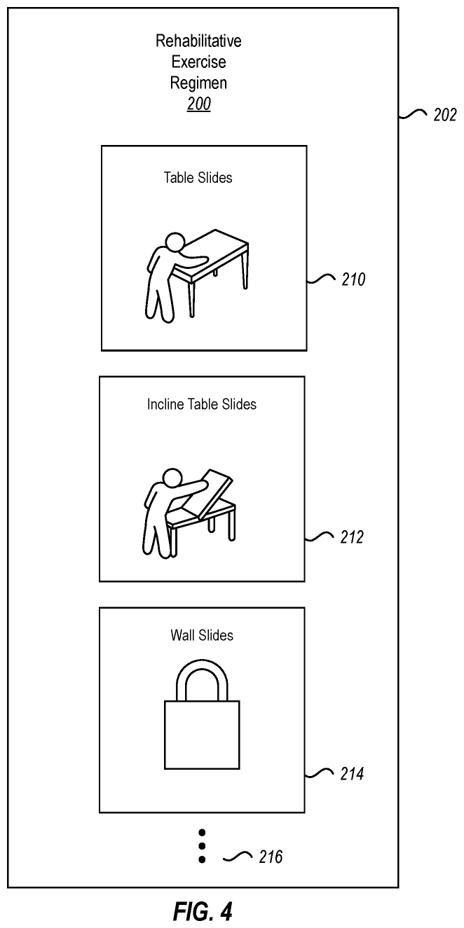

Block 320 of FIG. 3B (provided in bold format) illustrates that, in response to determining that the exercise analysis output 310 indicates satisfactory completion of the first rehabilitative exercise 210 by the user 302, the system 300 may permit the user 302 to proceed to the second rehabilitative exercise 212. For example, FIG. 4 illustrates the user interface 202 displaying the rehabilitative exercise regimen 200 with an additional rehabilitative exercise enabled. In particular, FIG. 4 illustrates an example in which the system 300 modifies the access state of the second rehabilitative exercise 212 from the locked state to an unlocked state, which is depicted by changing the lock symbol previously associated with the second rehabilitative exercise 212 (see FIG. 2) to a representation of the second rehabilitative exercise. The second rehabilitative exercise 212 may be selectable (e.g., on an exercise interface 304) to allow the user 302 to initiate and/or log progress toward completion of the second rehabilitative exercise 212 on the exercise interface 304. The system may also, in some instances, provide a “pass” notification to the user 302 via the exercise interface 304, indicating that the user 302 performed the first rehabilitative exercise 210 in a satisfactory manner.

As used herein, a “locked state” refers to any system configuration that is structured to prevent a user from accessing particular information and/or functionality (e.g., interacting with a rehabilitative exercise at an exercise interface, marking a rehabilitative exercise as complete, etc.), and, in contrast, an “unlocked state” refers to any system configuration that is structured to permit a user to access particular information and/or functionality.

One will appreciate, in view of the present disclosure, that satisfactory completion of a rehabilitative exercise may be determined based on exercise analysis output derived from any number of exercise sessions (e.g., one or more exercise sessions).

The functionality described above of preventing user access to a subsequent rehabilitative exercise until satisfactory completion of a preceding rehabilitative exercise is detected can prevent users from prematurely attempting advanced exercises that are beyond their physical capabilities. In this way, users undergoing a rehabilitative exercise regimen may avoid injury or slowed/ineffective rehabilitation that may result from incorrect performance of rehabilitative exercises and/or incorrect progression through a series of rehabilitative exercises.

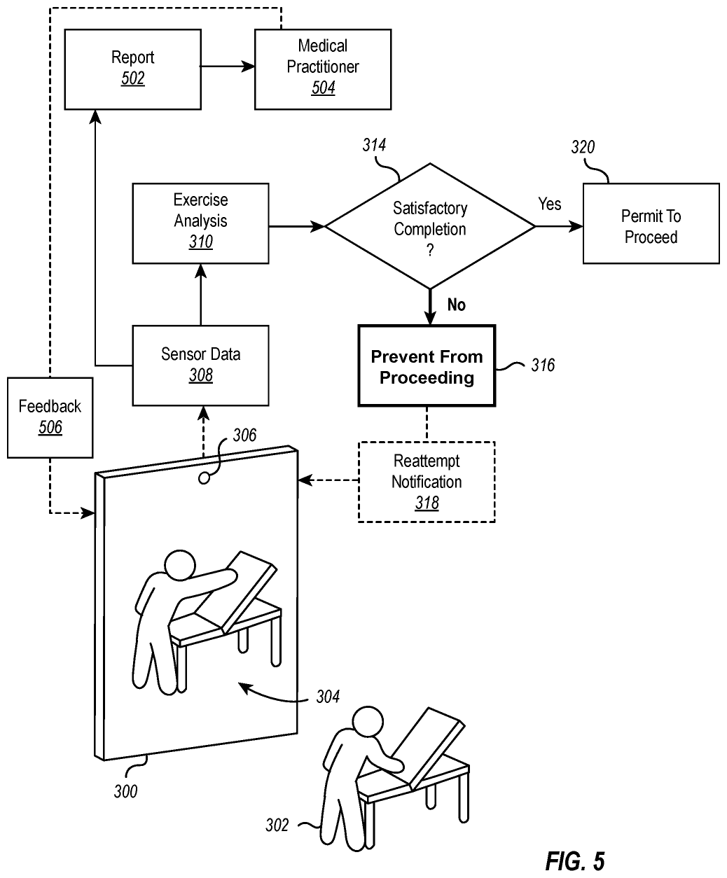

FIG. 5 illustrates a conceptual representation of the system 300 responding to the user 302 performing the second rehabilitative exercise 212 (i.e., incline table slides) in a manner that differs from an expected performance of the second rehabilitative exercise 212. FIG. 5 illustrates that, in some implementations, a report 502 may be generated based on the sensor data 308 that captures the user performance of the second rehabilitative exercise 212. The report 502 may, in some instances, be generated based on the exercise analysis output 310 (which is also generated based on the sensor data 308).

A report 502 may include various attributes of the exercise performance of the user 302 and/or indications of compliance of the user 302 with aspects of a rehabilitative exercise regimen. For example, the report 502 may include analysis metrics associated with the user's exercise performance (e.g., metrics describing bodily state, such as body positioning, heartrate, time spent in isometric positions, etc.) and/or associated with a comparison between the user's performance and an expected performance (e.g., metrics describing differences in form, repetitions, time spent in isometric positions, and/or others). In some implementations, a report 502 may include depictions of the user's exercise performance, such as video and/or images captured by the system 300 during the user's performance of the rehabilitative exercise (e.g., the second rehabilitative exercise 212).

Furthermore, in some implementations, a report 502 may include a live video feed of the user performing the rehabilitative exercise. For example, a user 302 initiating a rehabilitative exercise session may automatically trigger a remote video communication between the system 300 and a remote device, allowing a remote user to evaluate the user's performance of a rehabilitative exercise (e.g., the second rehabilitative exercise 212) in near real-time.

FIG. 5 illustrates that the report 502 may be provided to a medical practitioner 504. For example, a remote system associated with a medical practitioner 504 responsible for assisting in the rehabilitation of the user 302 may receive the report 502 and thereby have an opportunity to evaluate the user's progress toward complying with the rehabilitative exercise regimen 200. The medical practitioner 504 may thereby transmit feedback 506 to the system 300 (or another system) for viewing by the user to assist or encourage the user in complying with the rehabilitative exercise regimen. Such feedback 506 may be presented on the exercise interface 304 and may provide instruction to assist the user in performing rehabilitative exercises in a satisfactory manner. In some instances, feedback 506 takes the form of a video communication (e.g., near real-time remote video communication) between the user 302 and the medical practitioner 504 facilitated using the system 300 and the remote system associated with the medical practitioner 504.

In some instances, such functionality (e.g., receiving a report 502 and/or providing feedback 506) allows medical practitioners to analyze user exercise in a manner that complies with applicable medical procedures for tracking/facilitating user compliance with rehabilitative activities (indicated by current procedural terminology (CPT) codes), thereby advantageously allowing medical practitioners to provide and bill for medical services in a remote manner (e.g., reducing the risk of infectious disease transmission between the medical practitioner and the user).

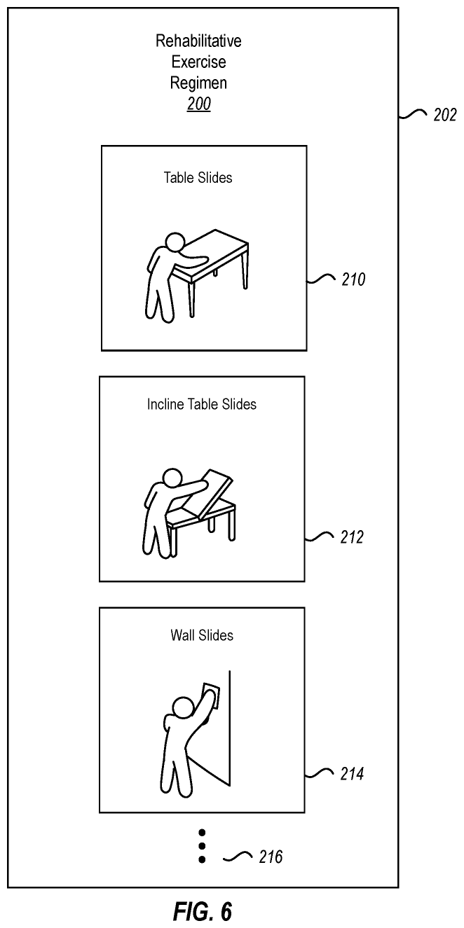

The user 302 may use the feedback to perform the second rehabilitative exercise 212 in a satisfactory manner, thereby allowing the user to proceed to a subsequent rehabilitative exercise. FIG. 6 illustrates the example user interface 202 displaying a representation of the third rehabilitative exercise 214 (i.e., wall slides) in an unlocked state, allowing the user 302 to access the third rehabilitative exercise 214.

The following discussion now refers to a number of methods and method acts that may be performed by the disclosed systems. Although the method acts are discussed in a certain order and illustrated in a flow chart as occurring in a particular order, no particular ordering is required unless specifically stated, or required because an act is dependent on another act being completed prior to the act being performed. One will appreciate that certain embodiments of the present disclosure may omit one or more of the acts described herein.

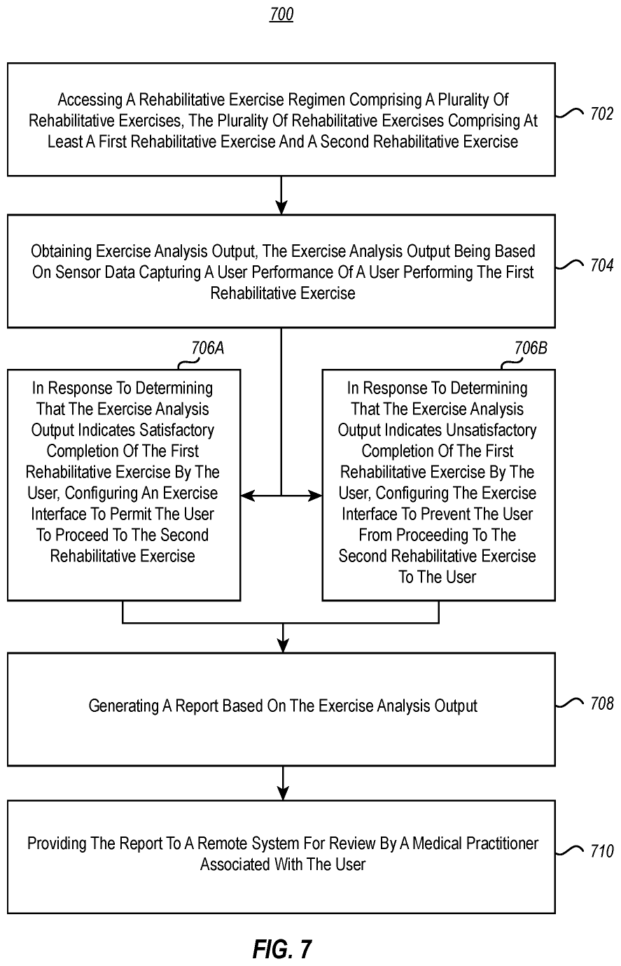

FIG. 7 illustrates an example flow diagram depicting acts associated with facilitating rehabilitative exercise compliance. The discussion of the various acts represented in the flow diagram 700 includes reference to various hardware components described in more detail with reference to FIG. 1.

Act 702 of flow diagram 700 includes accessing a rehabilitative exercise regimen comprising a plurality of rehabilitative exercises, the plurality of rehabilitative exercises comprising at least a first rehabilitative exercise and a second rehabilitative exercise. Act 702 is performed, in some instances, by a system 100 utilizing processor(s) 102, storage 104, sensor(s) 110, input/output system(s) 114, communication system(s) 116, and/or other components. In some implementations, each particular rehabilitative exercise of the plurality of rehabilitative exercises is associated with a respective rehabilitative progression level. The first rehabilitative exercise of the plurality of rehabilitative exercises is associated with a first rehabilitative progression level that is lower than a second rehabilitative progression level associated with the second rehabilitative exercise of the plurality of rehabilitative exercises.

Act 704 of flow diagram 700 includes obtaining exercise analysis output, the exercise analysis output being based on sensor data capturing a user performance of a user performing the first rehabilitative exercise. Act 704 is performed, in some instances, by a system 100 utilizing processor(s) 102, storage 104, sensor(s) 110, input/output system(s) 114, communication system(s) 116, and/or other components. In some implementations, the exercise analysis output comprises a comparison between the user performance and an expected performance of the first rehabilitative exercise. For example, the comparison may be based on a detected number of repetitions from the user performance and an expected number of repetitions associated with the expected performance. In some instances, the comparison is based on a detected amount of time in an isometric position from the user performance and an expected amount of time in the isometric position associated with the expected performance. The comparison may additionally or alternatively be based on a detected form from the user performance and an expected form associated with the expected performance.

Flow diagram 700 illustrates that various actions may be performed based on whether the exercise analysis output indicates satisfactory completion of the first rehabilitative exercise by the user (e.g., acts 706A and 706B).

Act 706A of flow diagram 700 includes, in response to determining that the exercise analysis output indicates satisfactory completion of the first rehabilitative exercise by the user, configuring an exercise interface to permit the user to proceed to the second rehabilitative exercise. Act 706A is performed, in some instances, by a system 100 utilizing processor(s) 102, storage 104, sensor(s) 110, input/output system(s) 114, communication system(s) 116, and/or other components. In some implementations, configuring the exercise interface to permit the user to proceed to the second rehabilitative exercise, in response to determining that the exercise analysis output indicates satisfactory completion of the first rehabilitative exercise by the user, includes configuring the exercise interface to modify an access state of the second rehabilitative exercise from a locked state to an unlocked state. Furthermore, in some instances, configuring the exercise interface to permit the user to proceed to the second rehabilitative exercise includes triggering display of a representation of the second rehabilitative exercise on the exercise interface.

Act 706B of flow diagram 700 includes, in response to determining that the exercise analysis output indicates unsatisfactory completion of the first rehabilitative exercise by the user, configuring the exercise interface to prevent the user from proceeding to the second rehabilitative exercise to the user. Act 706B is performed, in some instances, by a system 100 utilizing processor(s) 102, storage 104, sensor(s) 110, input/output system(s) 114, communication system(s) 116, and/or other components. In some implementations, configuring the exercise interface to prevent the user from proceeding to the second rehabilitative exercise, in response to determining that the exercise analysis output indicates unsatisfactory completion of the first rehabilitative exercise by the user, includes configuring the exercise interface to maintain an access state of the second rehabilitative exercise in a locked state or modifying an access state of the second rehabilitative exercise from an unlocked state to the locked state. Furthermore, in some instances, configuring the exercise interface to prevent the user from proceeding to the second rehabilitative exercise includes configuring the exercise interface to provide a notification to the user to reattempt the first rehabilitative exercise.

Regardless of whether acts 706A and/or 706B were performed, additional acts may be performed based on the exercise analysis output. Act 708 of flow diagram 700 includes generating a report based on the exercise analysis output. Act 708 is performed, in some instances, by a system 100 utilizing processor(s) 102, storage 104, sensor(s) 110, input/output system(s) 114, communication system(s) 116, and/or other components. The report may include an indication of compliance of the user with one or more portions of the rehabilitative exercise regimen.

Act 710 of flow diagram 700 includes providing the report to a remote system for review by a medical practitioner associated with the user. Act 710 is performed, in some instances, by a system 100 utilizing processor(s) 102, storage 104, sensor(s) 110, input/output system(s) 114, communication system(s) 116, and/or other components. The medical practitioner may review the user's exercise performance using the report and/or provide feedback to the user based on the report. In this way, in some instances, the medical practitioner is able to remotely perform billable medical services without face-to-face interaction with the user.

Disclosed embodiments may comprise or utilize a special purpose or general-purpose computer including computer hardware, as discussed in greater detail below. Disclosed embodiments also include physical and other computer-readable media for carrying or storing computer-executable instructions and/or data structures. Such computer-readable media can be any available media that can be accessed by a general-purpose or special-purpose computer system. Computer-readable media that store computer-executable instructions in the form of data are one or more “physical computer storage media” or “hardware storage device(s).” Computer-readable media that merely carry computer-executable instructions without storing the computer-executable instructions are “transmission media.” Thus, by way of example and not limitation, the current embodiments can comprise at least two distinctly different kinds of computer-readable media: computer storage media and transmission media.

Computer storage media (aka “hardware storage device”) are computer-readable hardware storage devices, such as RAM, ROM, EEPROM, CD-ROM, solid state drives (“SSD”) that are based on RAM, Flash memory, phase-change memory (“PCM”), or other types of memory, or other optical disk storage, magnetic disk storage or other magnetic storage devices, or any other medium that can be used to store desired program code means in hardware in the form of computer-executable instructions, data, or data structures and that can be accessed by a general-purpose or special-purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmission media can include a network and/or data links which can be used to carry program code in the form of computer-executable instructions or data structures and which can be accessed by a general purpose or special purpose computer. Combinations of the above are also included within the scope of computer-readable media.

Further, upon reaching various computer system components, program code means in the form of computer-executable instructions or data structures can be transferred automatically from transmission computer-readable media to physical computer-readable storage media (or vice versa). For example, computer-executable instructions or data structures received over a network or data link can be buffered in RAM within a network interface module (e.g., a “NIC”), and then eventually transferred to computer system RAM and/or to less volatile computer-readable physical storage media at a computer system. Thus, computer-readable physical storage media can be included in computer system components that also (or even primarily) utilize transmission media.

Computer-executable instructions comprise, for example, instructions and data which cause a general-purpose computer, special purpose computer, or special purpose processing device to perform a certain function or group of functions. The computer-executable instructions may be, for example, binaries, intermediate format instructions such as assembly language, or even source code.

Disclosed embodiments may comprise or utilize cloud computing. A cloud model can be composed of various characteristics (e.g., on-demand self-service, broad network access, resource pooling, rapid elasticity, measured service, etc.), service models (e.g., Software as a Service (“SaaS”), Platform as a Service (“PaaS”), Infrastructure as a Service (“IaaS”), and deployment models (e.g., private cloud, community cloud, public cloud, hybrid cloud, etc.).

Those skilled in the art will appreciate that the invention may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, pagers, routers, switches, wearable devices, and the like. The invention may also be practiced in distributed system environments where multiple computer systems (e.g., local and remote systems), which are linked through a network (either by hardwired data links, wireless data links, or by a combination of hardwired and wireless data links), perform tasks. In a distributed system environment, program modules may be located in local and/or remote memory storage devices.

Alternatively, or in addition, the functionality described herein can be performed, at least in part, by one or more hardware logic components. For example, and without limitation, illustrative types of hardware logic components that can be used include Field-programmable Gate Arrays (FPGAs), Program-specific Integrated Circuits (ASICs), Application-specific Standard Products (ASSPs), System-on-a-chip systems (SOCs), Complex Programmable Logic Devices (CPLDs), central processing units (CPUs), graphics processing units (GPUs), and/or others.

As used herein, the terms “executable module,” “executable component,” “component,” “module,” or “engine” can refer to hardware processing units or to software objects, routines, or methods that may be executed on one or more computer systems. The different components, modules, engines, and services described herein may be implemented as objects or processors that execute on one or more computer systems (e.g., as separate threads).

In some implementations, systems of the present disclosure may comprise or be configurable to execute any combination of software and/or hardware components that are operable to facilitate processing using machine learning models or other artificial intelligence-based structures/architectures. For example, one or more processors may comprise and/or utilize hardware components and/or computer-executable instructions operable to carry out function blocks and/or processing layers configured in the form of, by way of non-limiting example, single-layer neural networks, feed forward neural networks, radial basis function networks, deep feed-forward networks, recurrent neural networks, long-short term memory (LSTM) networks, gated recurrent units, autoencoder neural networks, variational autoencoders, denoising autoencoders, sparse autoencoders, Markov chains, Hopfield neural networks, Boltzmann machine networks, restricted Boltzmann machine networks, deep belief networks, deep convolutional networks (or convolutional neural networks), deconvolutional neural networks, deep convolutional inverse graphics networks, generative adversarial networks, liquid state machines, extreme learning machines, echo state networks, deep residual networks, Kohonen networks, support vector machines, neural Turing machines, and/or others.

Various alterations and/or modifications of the inventive features illustrated herein, and additional applications of the principles illustrated herein, which would occur to one skilled in the relevant art and having possession of this disclosure, can be made to the illustrated embodiments without departing from the spirit and scope of the invention as defined by the claims, and are to be considered within the scope of this disclosure. Thus, while various aspects and embodiments have been disclosed herein, other aspects and embodiments are contemplated. While a number of methods and components similar or equivalent to those described herein can be used to practice embodiments of the present disclosure, only certain components and methods are described herein.

It will also be appreciated that systems, devices, products, kits, methods, and/or processes, according to certain embodiments of the present disclosure may include, incorporate, or otherwise comprise properties, features (e.g., components, members, elements, parts, and/or portions) described in other embodiments disclosed and/or described herein. Accordingly, the various features of certain embodiments can be compatible with, combined with, included in, and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include said features, members, elements, parts, and/or portions without necessarily departing from the scope of the present disclosure.

Moreover, unless a feature is described as requiring another feature in combination therewith, any feature herein may be combined with any other feature of a same or different embodiment disclosed herein. Furthermore, various well-known aspects of illustrative systems, methods, apparatus, and the like are not described herein in particular detail in order to avoid obscuring aspects of the example embodiments. Such aspects are, however, also contemplated herein.

The present disclosure may be embodied in other specific forms without departing from its spirit or essential characteristics. The described embodiments are to be considered in all respects only as illustrative and not restrictive. The scope of the invention is, therefore, indicated by the appended claims rather than by the foregoing description. While certain embodiments and details have been included herein and in the attached disclosure for purposes of illustrating embodiments of the present disclosure, it will be apparent to those skilled in the art that various changes in the methods, products, devices, and apparatus disclosed herein may be made without departing from the scope of the disclosure or of the invention, which is defined in the appended claims. All changes which come within the meaning and range of equivalency of the claims are to be embraced within their scope.

What is claimed is:

1. A system for facilitating compliance with a rehabilitative exercise regimen, the system comprising:
one or more processors; and
one or more hardware storage devices storing instructions that are executable by the one or more processors to configure the system to:
access a rehabilitative exercise regimen comprising a plurality of rehabilitative exercises, the plurality of rehabilitative exercises comprising at least a first rehabilitative exercise and a second rehabilitative exercise;
obtain exercise analysis output, the exercise analysis output being based on sensor data capturing a user performance of a user performing the first rehabilitative exercise; and
in response to determining that the exercise analysis output indicates satisfactory completion of the first rehabilitative exercise by the user, configure an exercise interface to permit the user to proceed to the second rehabilitative exercise.

2. The system of claim 1, wherein each particular rehabilitative exercise of the plurality of rehabilitative exercises is associated with a respective rehabilitative progression level.

3. The system of claim 2, wherein the first rehabilitative exercise of the plurality of rehabilitative exercises is associated with a first rehabilitative progression level that is lower than a second rehabilitative progression level associated with the second rehabilitative exercise of the plurality of rehabilitative exercises.

4. The system of claim 1, wherein the exercise analysis output comprises a comparison between the user performance and an expected performance of the first rehabilitative exercise.

5. The system of claim 4, wherein the comparison is based on a detected number of repetitions from the user performance and an expected number of repetitions associated with the expected performance.

6. The system of claim 4, wherein the comparison is based on a detected amount of time in an isometric position from the user performance and an expected amount of time in the isometric position associated with the expected performance.

7. The system of claim 4, wherein the comparison is based on a detected form from the user performance and an expected form associated with the expected performance.

8. The system of claim 1, wherein configuring the exercise interface to permit the user to proceed to the second rehabilitative exercise, in response to determining that the exercise analysis output indicates satisfactory completion of the first rehabilitative exercise by the user, comprises configuring the exercise interface to modify an access state of the second rehabilitative exercise from a locked state to an unlocked state.

9. The system of claim 1, wherein configuring the exercise interface to permit the user to proceed to the second rehabilitative exercise, in response to determining that the exercise analysis output indicates satisfactory completion of the first rehabilitative exercise by the user, comprises triggering display of a representation of the second rehabilitative exercise on the exercise interface.

10. The system of claim 1, wherein the instructions are executable by the one or more processors to further configure the system to, in response to determining that the exercise analysis output indicates unsatisfactory completion of the first rehabilitative exercise by the user, configure the exercise interface to prevent the user from proceeding to the second rehabilitative exercise to the user.

11. The system of claim 10, wherein configuring the exercise interface to prevent the user from proceeding to the second rehabilitative exercise, in response to determining that the exercise analysis output indicates unsatisfactory completion of the first rehabilitative exercise by the user, comprises configuring the exercise interface to maintain an access state of the second rehabilitative exercise in a locked state or modifying an access state of the second rehabilitative exercise from an unlocked state to the locked state.

12. The system of claim 10, wherein configuring the exercise interface to prevent the user from proceeding to the second rehabilitative exercise, in response to determining that the exercise analysis output indicates unsatisfactory completion of the first rehabilitative exercise by the user, comprises configuring the exercise interface to provide a notification to the user to reattempt the first rehabilitative exercise.

13. The system of claim 1, wherein the instructions are executable by the one or more processors to further configure the system to:
generate a report based on the exercise analysis output; and
provide the report to a remote system for review by a medical practitioner associated with the user.

14. The system of claim 13, wherein the report comprises an indication of compliance of the user with one or more portions of the rehabilitative exercise regimen.

15. A method for facilitating compliance with a rehabilitative exercise regimen, the method comprising:
accessing a rehabilitative exercise regimen comprising a plurality of rehabilitative exercises, the plurality of rehabilitative exercises comprising at least a first rehabilitative exercise and a second rehabilitative exercise;
obtaining exercise analysis output, the exercise analysis output being based on sensor data capturing a user performance of a user performing the first rehabilitative exercise; and
in response to determining that the exercise analysis output indicates satisfactory completion of the first rehabilitative exercise by the user, configuring an exercise interface to permit the user to proceed to the second rehabilitative exercise.

16. The method of claim 15, wherein each particular rehabilitative exercise of the plurality of rehabilitative exercises is associated with a respective rehabilitative progression level.

17. The method of claim 16, wherein the first rehabilitative exercise of the plurality of rehabilitative exercises is associated with a first rehabilitative progression level that is lower than a second rehabilitative progression level associated with the second rehabilitative exercise of the plurality of rehabilitative exercises.

18. The method of claim 15, wherein the exercise analysis output comprises a comparison between the user performance and an expected performance of the first rehabilitative exercise.

19. The method of claim 15, wherein configuring the exercise interface to permit the user to proceed to the second rehabilitative exercise, in response to determining that the exercise analysis output indicates satisfactory completion of the first rehabilitative exercise by the user, comprises configuring the exercise interface to modify an access state of the second rehabilitative exercise from a locked state to an unlocked state.

20. One or more hardware storage devices storing instructions that are executable by one or more processors of a system to configure the system to:
access a rehabilitative exercise regimen comprising a plurality of rehabilitative exercises, the plurality of rehabilitative exercises comprising at least a first rehabilitative exercise and a second rehabilitative exercise;
obtain exercise analysis output, the exercise analysis output being based on sensor data capturing a user performance of a user performing the first rehabilitative exercise; and
in response to determining that the exercise analysis output indicates satisfactory completion of the first rehabilitative exercise by the user, configure an exercise interface to permit the user to proceed to the second rehabilitative exercise.
